(19) United States
(12) Patent Application Publication
(10) Pub. No.: US 2005/0113384 A1
(43) Pub. Date: May 26, 2005

(54) Combinations of (a) an atp-competitive inhibitor of c-acl kinase activity with (b) two or more other antineoplastic agents

(76) Inventor: Ioannis Alexander Avramis, Sylmar, CA (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/505,399

(22) PCT Filed: Feb. 27, 2003
(86) PCT No.: PCT/EP03/02029

Related U.S. Application Data
(60) Provisional application No. 60/360,197, filed on Feb. 28, 2002.

(51) Int. Cl. A61K 31/506

(52) U.S. Cl. 514/252.18

(57) ABSTRACT

The invention relates to combinations of (a) an ATP-competitive inhibitor of c-abl kinase activity with (b) two or more other antineoplastic agents for simultaneous, separate or sequential use, in particular for the delay of progression or treatment of a proliferative disease.

The invention relates to combinations of (a) an ATP-competitive Inhibitor of c-abl kinase activity with (b) two or more other antineoplastic agents for simultaneous, separate or sequential use, in particular for the delay of progression or treatment of a proliferative disease; to a method of treating a warm-blooded animal, especially a human, having a proliferative disease comprising administering to the animal a combination which comprises (a) an ATP-competitive inhibitor of c-abl kinase activity and (b) two or more other antineoplastic agents; a pharmaceutical composition comprising such a combination; the use of such a combination for the preparation of a medicament for the delay of progression or treatment of a proliferative disease; and to a commercial package or product comprising such a combination.

### BACKGROUND OF THE INVENTION

Single compound as well as selective combinations of purine and pyrimidine analogs are known to increase remission rates, especially in pediatric patients with relapsed leukaemias.

For example, Ara-C, a pyrimidine analog, is the 2′-alpha-hydroxy ribose (arabinoside) derivative of deoxycytidine. The anti-leukemic activity of this compound is long-established and it is an important agent in the treatment of both pediatric and adult patients with acute or chronic leukaemias as well as non-Hodgkin's lymphoma. 6-Mercaptopurine (6-MP) is a purine analog of hypoxanthine that competes with the latter for inosinic acid phosphorylase. The combination of 6-MP and ara-C decreases leukemic cell survival in acute myeloblastic leukaemia (AML) patients in a manner similar to that observed for fludarabine phosphate and ara-C.

Idarubicin (4-demethoxydaunorubicin), a topoisomerase II inhibitor, is also known to be useful in acute non-lymphocytic leukaemia, but also in the blast crisis of chronic granulocytic leukaemia or against acute lymphocytic leukaemia (ALL).

These and other chemotherapeutics are often used in, mainly pairwise, combination.

In addition, ATP-competitive Inhibitors of c-abl kinase activity have recently been found to be of use in the treatment of leukaemias, especially chronic myeloic leukaemia (CML). Treatment of CML formerly mainly included the use of hydroxyurea, α-interferon with or without ara-C or stem cell transplantation, were less than satisfactory because of patient intolerance or lack of effect on the natural history of this disorder.

In the vast majority of the CML cases, a characteristic t(9;22) translocation juxtaposes the 5′ end of the bcr gene with the 3′ end of the abl gene, resulting in a unique 210 kDa fusion protein p210^{bcr/abl 2-5} This constitutively active cytoplasmic kinase is capable of not only transforming murine fibroblasts and hematopoietic cell lines, but also causing a chronic myeloproliferative disorder resembling CML upon transduction into mouse marrow.

The presence of the p210^{bcr/abl} kinase in the vast majority of CML cases, coupled with evidence implicating this kinase in the pathogenesis of CML, made this fusion protein an attractive target for CML-directed therapy. Previous efforts identified multiple p210^{bcr/abl} kinase inhibitors.

The most widely studied p210^{bcr/abl} inhibitor is STI571 (formerly known as CGP 57148, chemical name:). The ATP-binding site-directed agent STI571 is in the meantime already being marketed, e.g. in the USA as a product under the tradename Gleevec®. This agent is a reversible inhibitor that occupies the ATP binding pocket of p210^{bcr/abl} and stabilizes the kinase in an inactive conformation. Preclinical studies demonstrated that STI571 also inhibits the kinase activities of c-abl, platelet-derived growth factor receptor and the c-kit receptor. Phase I studies showed that STI571 has impressive activity against chronic phase CML but more limited activity against p190^{bcr/abl}-expressing acute lymphocytic leukaemia and the blast crisis phase of CML. Additional preclinical and clinical studies of STI571, alone and in combination with conventional cytotoxic agents, are currently ongoing.

In view of the relatively high toxicities associated with the treatment of proliferative diseases, especially leukemnias, by chemotherapeutics such as those mentioned above, it remains a goal to devise novel treatment schedules or novel combinations that in principle allow for treatment with lower doses of the individual compounds, thus making it possible to allow for diminuation of the toxicities individually associated with highly toxic compounds. In addition, novel treatment regimens and combinations allowing for improved efficiency in the treatment of proliferative diseases remain an ever existing need. Furthermore, specific proliferative diseases and/or specific patient groups (e.g. related to sex or especially age, such as in case of pediatric or geriatric use, or patients where the proliferating cells became refractory to treatment with known chemotherapeutics or combinations thereof) may require more specific, even individual therapeutic regimens.

### GENERAL DESCRIPTION OF THE INVENTION

Surprisingly, it has now been found that the combination of (a) an ATP-competitive inhibitor of c-abl kinase activity with (b) two or more other antineoplastic agents for simultaneous, separate or sequential use, in particular for the delay of progression or treatment of a proliferative disease, shows many of the advantages mentioned above as desirable.

Unexpectedly, it has been found that the antineoplastic effect, i.e. especially the delay of progression or treatment of a proliferative disease, in particular the treatment of a tumor or more particularly of a leukaemia, of a combination as defined herein is greater than the effects that can be achieved with either type of combination partner alone, i.e. greater than the effects of a therapy using only component (a) or the two or more combination partners of component (b) as defined herein. A further benefit is that lower doses of the active ingredients can be used, for example, that the dosages need not only often be smaller but are also applied less frequently, or can be used in order to diminish the incidence of side-effects, thus allowing an improved quality of life, a decreased mortality and/or a dedreased morbidity. This is in accordance with the desires and requirements of the patients to be treated.

In particular, such combinations can be shown to be synergistic, thus allowing improved therapeutic efficiency and/or lower dosing of the individual components as compared with combinations of only two or more of the other antineoplastic agents (b).

### DESCRIPTION OF THE FIGURES

FIG. 1 shows the combination index (CI) plot for CEM/0 cells derived from the median effect plot of N-{5-[4-(4-methyl-piperazino-methyl)-benzoylamido]-2-methylphenyl}-4-(3-pyridyl)-2-pyrimidine-amine monomesylate salt (STI571) plus fludarabine plus ara-C when these drugs are considered to be mutually non-exclusive (the same curve is obtained in case of mutual exclusivity). The line at CI=1 represents additivity, below it synergism is found, above it antagonism. Fa (Fraction affected). Treatment is for 48 h, with STI571 given first and after 4 h Fludarabine and ara-C addition at 24 h.

FIG. 2 shows the combination index (CI) plot for CEM/0 cells derived from the median effect plot of N-{5-[4-(4-methyl-piperazino-methyl)-benzoylamido]-2-methylphenyl}-4-(3-pyridyl)-2-pyrimidine-amine monomesylate salt (STI571) plus fludarabine plus ara-C when these drugs are considered to be mutually non-exclusive (the same curve is obtained in case of mutual exclusivity). The line at CI=1 represents additivity, below it synergism is found, above it antagonism. Fa (Fraction affected). Treatment is for 48 h, with Fludarabine given first and then after 4 h STI571 and ara-C at 24 h.

FIG. 3 shows the combination index (CI) plot for CEM/0 cells (circles) or CEM/ara-C/I/ASNase-0.5-2 (triangles) derived from the median effect plot of N-{5-[4-(4-methyl-piperazino-methyl)-benzoylamido]-2-methylphenyl}-4-(3-pyridyl)-2-pyrimidine-amine monomesylate salt (STI571) plus fludarabine plus ara-C when these drugs are considered to be mutually non-exclusive (the same curve is obtained in case of mutual exclusivity). The line at CI=1 represents additivity, below it synergism is found, above it antagonism. Fa (Fraction affected). Treatment is for 48 h, with Fludarabine given first and then after 4 h STI571 and then at 24 h ara-C.

FIG. 4 FIG. 1 shows the combination index (CI) plot for CEM/0 cells derived from the median effect plot of N-{5-[4-(4-methyl-piperazino-methyl)-benzoylamido]-2-methylphenyl}-4-(3-pyridyl)-2-pyrimidine-amine monomesylate salt (STI571) plus Idarubicin plus ara-C when these drugs are considered to be mutually non-exclusive (the same curve is obtained in case of mutual exclusivity). The line at CI=1 represents additivity, below it synergism is found, above it antagonism. Fa (Fraction affected). Treatment is for 72 h, with STI571 given first and after 4 h Idarubicin and at 24 h ara-C addition.

### DETAILED DESCRIPTION OF THE INVENTION

In one preferred embodiment, the invention relates to a combination of (a) an ATP-competitive Inhibitor of c-abl kinase activity with (b) two or more other antineoplastic agents for simultaneous, separate or sequential use, in particular for use in the delay of progression or the treatment of a proliferative disease in a warm-blooded animal, especially a human.

In another preferred embodiment, the invention relates to a method of treating a warm-blooded animal, especially a human, suffering from a proliferative disease, comprising administering to said animal a combination which comprises (a) an ATP-competitive inhibitor of c-abl kinase activity and (b) two or more other antineoplastic agents, preferably in such a way that the components (a) and (b) are jointly therapeutically active in the treatment of said disease; in particular in a dose that is pharmaceutically effective in the treatment of said disease.

A further embodiment of the Invention relates to a pharmaceutical composition comprising a combination of (a) an ATP-competitive inhibitor of c-abl kinase activity with (b) two or more other antineoplastic agents and optionally at least one pharmaceutically acceptable carrier, preferably for simultaneous, separate or sequential use, especially in the delay of progression or treatment of a proliferative disease in a warm-blooded animal, especially a human, requiring such treatment.

Still a further embodiment of the invention relates to the use of a combination of (a) an ATP-competitive inhibitor of c-abl kinase activity with (b) two or more other antineoplastic agents for simultaneous, sequential or separate use, for the delay of progression or the treatment of a proliferative disease; and/or for the manufacture of a pharmaceutical preparation for the delay of progression or treatment of said disease.

Yet another embodiment of the invention relates to a commercial package or product comprising (a) an ATP-competitive inhibitor of c-abl kinase activity and (b) two or more other anti-neoplastic agents for simultaneous, chronically staggered or (less preferably) separate use, especially for the delay of progression or the treatment of a proliferative disease.

The general terms used hereinbefore and hereinafter preferably have within the context of this disclosure the following meanings, unless otherwise indicated:

As components (a) and (b), the following are very preferred:

Component (a): An ATP-competitive inhibitor of c-abl kinase activity is preferably a low molecular weight (Mᵣ<1500) inhibitor c-abl kinase, especially of the p210^{bcr/abl} 210 kDa fusion protein, or a pharmaceutically acceptable salt thereof, especially of the 2-phenylaminopyrimidine class, preferably a compound as described in EP 0 564 409, most preferably (N-{5-[4-(4-methyl-piperazino-methyl)-benzoylamido]-2-methylphenyl}-4-(3-pyridyl)-2-pyrimidine-amine, especially in the form of the methane sulfonate (monomesylate) salt (“STI571” hereinafter); or in a broader aspect of the invention of the 2-thiophen-quinoxaline class, preferably 6,7-dimethoxy-2-thiophen-3-yl-quinoxaline, especially in the form of the hydrochloride salt (RPR101511A), which may also Inhibit the secretion of VEGF, PDGF, EGF and related epithelium secreted growth factors.

The inhibition of c-abl kinase, especially e.g. bcr/abl kinase can be determined according to methods known in the art (see, e.g., Nature Medicine 2, 561-566 (1996), or Gombacorti et al., Blood Cells, Molecules and Diseases 23, 380-394 (1997)), allowing to Identify c-abl kinase inhibitors.

Component (b): Component (b) preferably comprises two or more, more preferably two or three, most preferably two antineoplastic agents other than component (a), in the latter case leading to a triple drug combination.

The term “antineoplastic agents” as used herein includes, but is not limited to aromatase inhibitors, antiestrogens, topoisomerase I inhibitors, microtubule active agents, alkylating agents, antineoplastic antimetabolites, platin compounds, compounds decreasing the protein kinase activity or further anti-anglogenic compounds, gonadorelin agonists, anti-androgens, bisphosphonates and trastuzumab, ribonucleotide reducatase inhibitors, preferably topoisomerase II inhibitors or pyrimidine or purine nucleoside analogs.

The term “pyrimidine or purine nucleoside analogs” as used herein includes, but is not limited to fludarabine and/or cytosine arabinoside (ara-C) (which are preferred), but also 6-thioguanine, 5-fluorouracil, cladribine, 6-mercaptopurine (especially in combination with ara-C against ALL) and/or pentostatin.

The term “topoisomerase II inhibitors” as used herein includes, but is not limited to the antracyclines doxorubicin, epirubicin, idarubicin and nemorubicin, the anthraquinones mitoxantrone and Iosoxantrone, and the podophyllotoxines etoposide and teniposide. Etoposide can be administered, e.g., in the form as it is marketed, e.g. under the trademark ETOPOPHOS™. Teniposide can be administered, e.g., in the form as it is marketed, e.g. under the trademark VM 26-BRISTOL™. Doxorubicin can be administered, e.g., in the form as it is marketed, e.g. under the trademark ADRIBLASTIN™. Epirubicin can be administered, e.g., in the form as it is marketed, e.g. under the trademark FARMORUBICIN™. Idarubicin can be administered, e.g., in the form as it is marketed, e.g. under the trademark ZAVEDOS™. Mitoxantrone can be administered, e.g., in the form as it is marketed, e.g. under the trademark NOVANTRON™. Idarubicin is preferred.

The term “aromatase inhibitors” as used herein relates to compounds which inhibit the estrogen production, i.e. the conversion of the substrates androstenedione and testosterone to estrone and estradiol, respectively. The term includes, but is not limited to steroids, especially exemestane and formestane and, in particular, non-steroids, especially aminoglutethimide, vorozole, fadrozole, anastrozole and, very especially, letrozole.

The term “antiestrogens” as used herein relates to compounds which antagonize the effect of estrogens at the estrogen receptor level. The term includes, but is not limited to tamoxifen, fulvestrant, raloxifene and raloxifene hydrochloride.

The term “topoisomerase I inhibitors” as used herein includes, but is not limited to topotecan, irinotecan, 9-nitrocamptothecin and the macromolecular camptothecin conjugate PNU-166148 (compound A1 in WO99/17804).

The term “microtubule active agents” relates to microtubule stabilizing and microtubule destabilizing agents including, but not limited to the taxanes paclitaxel and docetaxel, the vinca alkaloids, e.g., vinblastine, especially vinblastine sulfate, vincristine especially vincristine sulfate, and vinorelbine, discodermolide and epothilones. Discodermolide can be obtained, e.g., as disclosed in U.S. Pat. No. 5,010,099.

The term “alkylating agents” as used herein includes, but is not limited to cyclophosphamide, ifosfamide and melphalan.

The term “antineoplastic antimetabolites” includes, but is not limited to 5-fluorouracil, capecitabine, gemcitabine, methotrexate and edatrexate.

The term “platin compounds” as used herein includes, but is not limited to carboplatin, cisplatin and oxaliplatin.

The term “compounds decreasing the protein kinase activity and further anti-angiogenic compounds” as used herein includes, but is not limited to compounds decreasing the activity of the epidermal growth factor (EGF) of the epidermal growth factor (EGF), the vascular endothelial growth factor (VEGF), the platelet derived growth factor (PDGF) and/or the protein kinase C and anti-angiogenic compounds having another mechanism for their activity. Preferably, the term relates to inhibitors of protein kinase activity other than c-abl kinase activity or to tyrphostins, in the latter case preferably with the proviso that at least one of the other antineoplastic agents is other than a c-abl inhibitor or a tyrphostin.

Compounds which decreases the activity of VEGF are especially compounds which Inhibit the VEGF receptor tyrosine kinase, compounds which inhibit a VEGF receptor and compounds binding to VEGF, and are in particular those compounds, proteins and monoclonal antibodies generically and specifically disclosed in WO 98/35958, WO 00/09495, WO 00/27820, WO 00/59509, WO 98/11223, WO 00/27819 and EP 0 769 947; those as described by M. Prewett et al in Cancer Research 59 (1999) 5209-5218, by F. Yuan et al in Proc. Natl. Acad. Sci. USA, vol. 93, pp. 14765-14770, December 1996, by Z. Zhu et al in Cancer Res. 58, 1998, 3209-3214, and by J. Mordenti et al in Toxicologic Pathology, Vol. 27, no. 1, pp 14-21, 1999; in WO 00/37502 and WO 94/10202; Angiostatin™, described by M. S. O'Reilly et al, Cell 79, 1994, 315-328; and Endostatin™, described by M. S. O'Reilly et al, Cell 88, 1997, 277-285;
compounds which decrease the activity of the epidermal growth factor (EGF) are especially compounds which inhibit the EGF receptor tyrosine kinase, compounds which inhibit the EGF receptor and compounds binding to EGF, and are in particular those compounds generically and specifically disclosed in WO 97/02266, EP 0 564 409, WO 99/03854, EP 0520722, EP 0 566 226, EP 0 787 722, EP 0 837 063, U.S. Pat. No. 5,747,498, WO 98/10767, WO 97/30034, WO 97/49688, WO 97/38983 and, especially, WO 96/33980; or
compounds which decreases the activity of the protein kinase C are especially those staurosporine derivatives disclosed in EP 0 296 110 (pharmaceutical preparation described in WO 00/48571) which compounds are protein kinase C inhibitors.

A tyrphostin is preferably a low molecular weight (Mᵣ<1500) compound, or a pharmaceutically acceptable salt thereof, especially a compound selected from the benzylidenemalonitrile class or the S-arylbenzenemalonirile or bisubstrate quinoline class of compounds (see Levitzki, FASEB J. 6, 3275-82 (1992)), more especially any compound selected from the group consisting of Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556 (see Levitsky et al., TiPS 12, 171 (1991; Ohmichi, Biochem. 32, 4650 (1993); Gazit et al., J. Med. Chem. 32, 2344; Levitski et al., Science 267, 1782 (1995); Gazit et al., J. Med. Chem. 39, 4905 (1996); Gazit et al., J. Med. Chem. 34, 189 (1991); Wang et al., J. Immunol. 162, 3897 (1999)), and especially AG957 of the formula 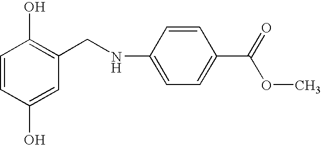
and most especially adaphostin (4-{[(2,5-dihydroxyphenyl)methyl]amino}-benzoic acid adamantyl ester; NSC 680410, Adaphostin) of the formula 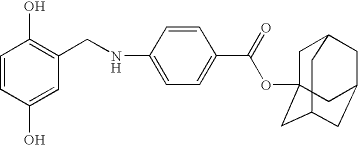
or (in case of each of the mentioned compounds, where salt-forming griups are present) a salt thereof.

In each case where citations of patent applications or scientific publications are given, in particular with regard to the respective compound claims and the final products of the working examples therein, the subject-matter of the final products, the pharmaceutical preparations and the claims is hereby incorporated into the present application by reference to these publications. Comprised are likewise the corresponding stereoisomers as well as the corresponding crystal modifications, e.g. solvates and polymorphs, which are disclosed therein. The compounds used as active ingredients in the combinations disclosed herein can be prepared and administered as described in the cited documents, respectively.

Further anti-angiogenic compounds are thalidomide (THALOMID), SU5416, and celecoxib (Celebrex).

The term “gonadorelin agonist” as used herein includes, but is not limited to abarelix, goserelin and goserelin acetate.

The term “anti-androgens” as used herein includes, but is not limited to bicalutamide.

The term “bisphosphonates” as used herein includes, but is not limited to etridonic acid, clodronic acid, tiludronic acid, pamidronic acid, alendronic acid, ibandronic acid, risedronic acid and zoledronic acid.

“Trastuzumab” can be administered, e.g., in the form as it is marketed, e.g. under the trademark HERCEPTIN™.

Ribonucleotide reductase inhibitors are especially hydroxyurea or 2-hydroxy-1H-isoindole-1,3-dione derivatives, such as PL-1. PL-2, PL-3, PL-4, PL-5, PL-6, PL-7 or PL-8 mentioned in P. Nandy et al., Acta Oncologica 33(8), 953-961 (1994).

The structure of the active agents identified by code nos., generic or trade names may be taken from the actual edition of the standard compendium “The Merck Index” or from databases, e.g. Patents International (e.g. IMS World Publications), or the publications mentioned above and below. The corresponding content thereof is hereby incorporated by reference.

It will be understood that references to the components (a) and (b) are meant to also include the pharmaceutically acceptable salts of any of the active substances (c-abl kinase inhibitor or antineoplastic agent) comprised. If active substances comprised by components (a) and/or (b) have, for example, at least one basic center, they can form acid addition salts. Corresponding acid addition salts can also be formed having, if desired, an additionally present basic center. Active substances having an acid group (for example COOH) can form salts with bases. The active substances comprised in components (a) and/or (b) or a pharmaceutically acceptable salts thereof may also be used in form of a hydrate or include other solvents used for crystallization. N-{5-[4-(4-methyl-piperazino-methyl)-benzoylamido]-2-methylphenyl}-4-(3-pyridyl)-2-pyrimidine-amine, i.e. the most preferred combination partner (a), is preferably used in the present invention in the form of its monomesylate salt (STI571).

Proliferative disease is especially a leukaemia or a lymphoma, preferably an acute leukaemia or the acute phase of a chronic leukaemia, especially an acute T-lymphoblastic leukaemia, but may also relate to a solid tumor which expresses VEGF and related growth factors and hence depends on autocrine growth loops, such as human glioblastoma, human medulloblastoma, and/or related solid tumors originating from neuronal crest derived cells, organs or tissues that secrete VEGF, PDGF, EGF and/or related growth factors.

The term “solid tumor” especially means breast cancer, cancer of the colon and generally the GI tract, lung cancer, in particular small-cell lung cancer, and non-small-cell lung cancer, head and neck cancer, genitourinary cancer, e.g. cervical, uterine, ovarian, testicles, prostate or bladder cancer; Hodgkin's disease or Kaposi's sarcoma. Depending on the tumor type and the particular combination used a decrease of the tumor volume can be obtained. The combinations disclosed herein are also suited to prevent the metastatic spread of tumors and the growth or development of micrometastases.

Simultaneous administration may, for example, take place in the form of one fixed combination with two or more active ingredients, or by simultaneously administering two or more active ingredients that are formulated independently. Sequential use (administration) preferably means administration of one (or more) components of a combination at one time point, other components at a different time point, that is, in a chronically staggered manner, preferably such that the combination shows more efficiency than the single compounds administered independently (especially showing synergism). Separate use (administration) preferably means administration of the components of the combination independently of each other at different time points, preferably meaning that the components (a) and (b) are administered such that no overlap of measureable blood levels of both compounds are present in an overlapping manner (at the same time).

Also combinations of two or more of sequential, separate and simultaneous administration are possible, preferably such that the combination component-drugs show a joint therapeutic effect that exceeds the effect found when the combination component-drugs are used independently at time intervals so large that no mutual effect on their therapeutic efficiency can be found, a synergistic effect being especially preferred.

Accordingly, the preparations according to the inventions may be fixed combinations of (a) a c-able kinase inhibitor and (b) two or more other antineoplastic agents, or combinations of more than one separate pharmaceutical preparations each comprising one (or more than one) of these active ingredients in separate form (e.g. in the sense of a kit of parts).

In a yet further aspect, the present invention provides a pharmaceutical preparation comprising a (a) a c-able kinase inhibitor and (b) two or more other antineoplastic agents, together with a pharmaceutically acceptable carrier.

The term “delay of progression” as used herein means administration of the combination to patients being in a pre-stage or in an early phase, of the first manifestation or a relapse of the disease to be treated, in which patients for example a pre-form of the corresponding disease diagnosed or which patients are in a condition, e.g. during a medical treatment or a condition resulting from an accident, under which it is likely that a corresponding disease will develop.

“Jointly therapeutically active” means that the compounds may be given separately (in a chronically staggered manner, especially a sequence-specific manner) in such time intervals that they preferably, in the warm-blooded animal, especially human, to be treated, still show a (preferably synergistic) interaction (joint therapeutic effect). Whether this is the case, can inter alia be determined by following the blood levels, showing that both compounds are present in the blood of the human to be treated at least during certain time intervals.

“Pharmaceutically effective” preferably relates to an amount that is therapeutically or in a broader sense also prophylactically effective against the progression of a proliferative disease, especially a leukaemia, preferably one as defined above. “Pharmaceutically effective” drug combinations are especially those resulting in a prolongation of complete remission (CR) of disease in patients with bone marrow and lymphoproliferative diseases, such as leukemias and lymphomas.

The term “a commercial package” or “a product”, as used herein defines especially a “kit of parts” in the sense that the components (a) and (b) as defined above can be dosed independently or by use of different fixed combinations with distinguished amounts of the components (a) and (b), i.e., simultaneously or at different time points. Moreover, these terms comprise a commercial package comprising (especially combining) as active ingredients components (a) and (b), together with instructions for simultaneous, sequential (chronically staggered, in time-specific sequence, preferentially) or (less preferably) separate use thereof in the delay of progression or treatment of a proliferative disease. The parts of the kit of parts can then, e.g., be administered simultaneously or chronologically staggered, that is at different time points and with equal or different time intervals for any part of the kit of parts. Very preferably, the time intervals are chosen such that the effect on the treated disease in the combined use of the parts is larger than the effect which would be obtained by use of only any one of the combination partners (a) and (b) (as can be determined according to standard methods, e.g. the determination of Combination Index or the use of isobolograms as described in the examples). The ratio of the total amounts of the combination partner (a) to the combination partner (b) to be administered in the combined preparation can be varied, e.g. in order to cope with the needs of a patient sub-population to be treated or the needs of the single patient which different needs can be due to the particular disease, age, sex, body weight, etc. of the patients. Preferably, there is at least one beneficial effect, e.g., a mutual enhancing of the effect of the combination partners (a) and (b), in particular a more than additive effect, which hence could be achieved with lower doses of each of the combined drugs, respectively, than tolerable in the case of treatment with the individual drugs only without combination, producing additional advantageous effects, e.g. less side effects or a combined therapeutical effect in a non-effective dosage of one or both of the combination partners (components) (a) and (b), and very preferably a strong synergism (Combination Index above 4) of the combination partners (a) and (b).

Both in the case of the use of the combination of components (a) and (b) and of the commercial package, any combination of simultaneous, sequential and separate use is also possible, meaning that the components (a) and (b) may be administered at one time point simultaneously, followed by administration of only one component with lower host toxicity either chronically (e.g. more than 3 to 4 weeks of daily dosing) at a later time point and subsequently the other component or the combination of both components at a still later time point (in subsequent drug combination treatment courses for an optimal antitumor effect) or the like.

Any of the combination of components (a) and (b), the method of treating a warm-blooded animal comprising administering these two components, a pharmaceutical composition comprising these two components for simultaneous, separate or sequential use, the use of the combination for the delay of progression or the treatment of a proliferative disease or for the manufacture of a pharmaceutical preparation for these purposes or a commercial product comprising such a combination of components (a) and (b), all as mentioned or defined above, will be referred to subsequently also as COMBINATION OF THE INVENTION (so that this term refers to each of these embodiments which thus can replace this term where appropriate).

It can be shown by established test models and in particular those test models described herein, e.g. in the Examples, that a COMBINATION OF THE INVENTION results in a more effective delay of progression or treatment of a proliferative disease compared to the effects observed with the single combination partners or combination according to component (b) only (two or more antineoplastic agents other than c-abl kinase inhibitors). The person skilled in the pertinent art is fully enabled to select a relevant test model to prove the therapeutic indications and beneficial effects hereinbefore and hereinafter mentioned. The pharmacological activity of a COMBINATION OF THE INVENTION may, for example, be demonstrated in a clinical study or in a test procedure as essentially described hereinafter.

Suitable clinical studies are, for example, open label non-randomized, dose escalation studies (Phase I) in patients with advanced solid tumors. Such studies prove (A) safety and (B) the synergism of the active ingredients of the COMBINATIONS OF THE INVENTION. The beneficial effects on proliferative diseases can be determined directly through the results of these studies or by changes in the study design which are known as such to a person skilled in the art. Such studies are, in particular, suitable to compare the effects of a monotherapy or a therapy using only two or more antineoplastic agents other than c-abl kinase inhibitors (component (b)) versus a COMBINATION OF THE INVENTION. Preferably, the combination partner (a) is administered with a fixed dose and the dose of the combination partner (b) is escalated until the Maximum Tolerated Dosage of the combination regimen is reached, or vice versa. In a preferred embodiment of the study, each patient receives daily doses of the combination partner (a). The efficacy of the treatment can be determined in such studies, e.g., after 4 to 8 weeks by evaluation of the status of the proliferative disease, in case of a leukaemia e.g. by determination of the count of aberrant white blood cells, and by staining mononuclear cells and/or by means of determining minimum residual disease (MRD) e.g. by FACS-LPC MRD or PCR. Alternatively, a placebo-controlled, double blind study can be used in order to prove the benefits of the COMBINATION OF THE INVENTION mentioned herein, once the safety of the treatment regimen(s) has been established.

The COMBINATION OF THE INVENTION can also be applied in combination with other treatments, e.g. surgical intervention, hyperthermia and/or irradiation therapy.

### PREFERRED EMBODIMENTS OF THE INVENTION

In the following preferred embodiments of the invention, more general terms can be replaced independently or totally by the more specific definitions given above, thus leading to still more preferred embodiments of the invention.

A COMBINATION OF THE INVENTION which comprises (a) N-{5-[4-(4-methyl-piperazino-methyl)-benzoylamido]-2-methylphenyl}-4-(3-pyridyl)-2-pyrimidine-amine, or a pharmaceutically acceptable salt thereof, and (b) at least two further antineoplastic agents, independently in free form or as pharmaceutically acceptable salts, preferably as defined above, is preferred.

More preferred is a COMBINATION OF THE INVENTION comprising (a) N-{5-[4-(4-methyl-piperazino-methyl)-benzoylamido]-2-methylphenyl}-4-(3-pyridyl)-2-pyrimidine-amine, or a pharmaceutically acceptable salt thereof, and (b) two or three, preferably two, further antineoplastic agents selected from purine nucleoside analogs and topolsomerase II inhibitors, independently in free form or as pharmaceutically acceptable salts.

Still more preferred is a COMBINATION OF THE INVENTION comprising (a) N-{5-[4-(4-methyl-piperazino-methyl)-benzoylamido]-2-methylphenyl}-4-(3-pyridyl)-2-pyrimidine-amine, or a pharmaceutically acceptable salt thereof, and (b) two further antineoplastic agents selected from Idarubicine, Fludarabine and ara-C, independently in free form or as pharmaceutically acceptable salts.

Most preferably the invention relates to a COMBINATION OF THE INVENTION comprising (a) N-{-[4-(4-methyl-piperazino-methyl)-benzoylamido]-2-methylphenyl}-4-(3-pyridyl)-2-pyrimidine-amine, or a pharmaceutically acceptable salt thereof, and (b) two further antineoplastic agents, especially selected from purine nucleoside analogs and topoisomerase II inhibitors, most especially selected from Idarubicin, Fludarabine and ara-C, independently in free form or as pharmaceutically acceptable salts, where the combination is such that administration of component (a) is started before administration of component (b), especially 2 to 48 hours before.

In any of the preceeding paragraphs describing preferred embodiments of the invention, those COMBINATIONS OF THE INVENTION are most preferred where the active compounds used in component (a) and component (b) are formulated independently or in the form of a kit of parts, in both cases based on pharmaceutical preparations that are already (e.g. commercially) available.

Pharmaceutical Preparations and Methods

The pharmaceutical preparations comprising component (a) and/or component (b), in the case of component (b) a fixed combination of the antineoplastic agents comprised therein or independent formulations for one or more of these antineoplastic agents for combined use, can be standard preparations of these components as already known in the art.

The pharmaceutical compositions comprise from about 0.00002 to about 95%, especially (e.g. in the case of infusion dilutions that are ready for use) of 0.0001 to 0.02%, or (for example in case of injection or infusion concentrates or especially parenteral formulations) from about 0.1% to about 95%, preferably from about 1% to about 90%, active ingredient (weight by weight, in each case). Pharmaceutical compositions according to the invention may be, for example, in unit dose form, such as in the form of ampoules, vials, dragees, tablets, infusion bags or capsules.

The effective dosage of each of the combination partners employed in the COMBINATION OF THE INVENTION may vary depending on the particular compound or pharmaceutical composition employed, the mode of administration, the condition being treated, the severity of the condition being treated. Thus, the dosage regimen the COMBINATION OF THE INVENTION is selected in accordance with a variety of factors including the route of administration and the renal and hepatic function of the patient. A physician, clinician or veterinarian of ordinary skill can readily determine and prescribe the effective amount of the single active ingredients required to prevent, counter or arrest the progress of the condition. Optimal precision in achieving concentration of the active ingredients within the range that yields efficacy without toxicity requires a regimen based on the kinetics of the active ingredients' availability to target sites.

The c-abl kinase inhibitors and further (other) antineoplastic agents forming part of components (a) and (b) are named “active ingredients” in the following definition of pharmaceutical preparations/compositions:

The pharmaceutical compositions of the present invention are prepared in a manner known per se, for example by means of conventional dissolving, lyophilizing, mixing, granulating or confectioning processes and combination with appropriate carrier materials.

Solutions of the active ingredient, and also suspensions, and especially isotonic aqueous solutions or suspensions, are useful for parenteral administration of the active ingredient, it being possible, for example in the case of lyophilized compositions that comprise the active ingredient alone or together with a pharmaceutically acceptable carrier, for example mannitol, for such solutions or suspensions to be produced prior to use. The pharmaceutical compositions may be sterilized and/or may comprise excipients, for example preservatives, stabilizers, wetting and/or emulsifying agents, solubilizers, salts for regulating the osmotic pressure and/or buffers, and are prepared in a manner known per se, for example by means of conventional dissolving or lyophilizing processes. The solutions or suspensions may comprise viscosity-increasing substances, such as sodium carboxymethylcellulose, carboxymethylcellulose, dextran, polyvinylpyrrolidone or gelatin. Suspensions in oil comprise as the oil component the vegetable, synthetic or semi-synthetic oils customary for injection purposes.

The injection or infusion compositions are prepared in customary manner under sterile conditions; the same applies also to introducing the compositions into ampoules or vials and sealing the containers.

An infusion solution preferably must have the same or essentially the same osmotic pressure as body fluid. Accordingly, the aqueous medium preferably contains an isotonic agent which has the effect of rendering the osmotic pressure of the infusion solution the same or essentially the same as body fluid.

The isotonic agent may be selected from any of those known in the art, e.g. mannitol, dextrose, glucose and sodium chloride. The infusion formulation may be diluted with the aqueous medium. The amount of aqueous medium employed as a diluent is chosen according to the desired concentration of active ingredient in the infusion solution.

Infusion solutions may contain other excipients commonly employed in formulations to be administered intravenously. Excipients include antioxidants. Infusion solutions may be prepared by mixing an ampoule or vial of the formulation with the aqueous medium, e.g. a 5% w/v glucose solution in WFI or especially 0.9% sodium chloride solution in a suitable container, e.g. an infusion bag or bottle. The infusion solution, once formed, is preferably used immediately or within a short time of being formed, e.g. within 6 hours. Containers for holding the infusion solutions may be chosen from any conventional container which is nonreactive with the infusion solution. Glass containers made from those glass types aforementioned are suitable although it may be preferred to use plastics containers, e.g. plastics infusion bags.

Pharmaceutical compositions for parenteral, e.g. oral administration, can be obtained by combining the active ingredient with solid carriers, if desired granulating a resulting mixture, and processing the mixture, if desired or necessary, after the addition of appropriate excipients, into tablets, dragee cores or capsules, or be filled into a powder inhalator for administration by inhalation. It is also possible for them to be incorporated into plastics carriers that allow the active ingredients to diffuse or be released in measured amounts.

Suitable carriers are especially fillers, such as sugars, for example lactose, saccharose, mannitol or sorbitol, cellulose preparations, and/or calcium phosphates, for example tricalcium phosphate or calcium hydrogen phosphate, and also binders, such as starches, for example corn, wheat, rice or potato starch, methylcellulose, hydroxypropyl methylcellulose, sodium carboxymethylcellulose, and/or polyvinylpyrrolidone, and/or, if desired, disintegrators, such as the above-mentioned starches, also carboxymethyl starch, crosslinked polyvinylpyrrolidone, alginic acid or a salt thereof, such as sodium alginate. Additional excipients are especially flow conditioners and lubricants, for example silicic acid, talc, stearic acid or salts thereof, such as magnesium or calcium stearate, and/or polyethylene glycol, or derivatives thereof.

Tablet cores can be provided with suitable, optionally enteric, coatings through the use of, inter alia, concentrated sugar solutions which may comprise gum arabic, talc, polyvinylpyrrolidone, polyethylene glycol and/or titanium dioxide, or coating solutions in suitable organic solvents or solvent mixtures, or, for the preparation of enteric coatings, solutions of suitable cellulose preparations, such as acetylcellulose phthalate or hydroxypropylmethylcellulose phthalate. Dyes or pigments may be added to the tablets or tablet coatings, for example for identification purposes or to indicate different doses of active ingredient.

Pharmaceutical compositions for oral administration also include hard capsules consisting of gelatin, and also soft, sealed capsules consisting of gelatin and a plasticizer, such as glycerol or sorbitol. The hard capsules may contain the active ingredient in the form of granules, for example in admixture with fillers, such as corn starch, binders, and/or glidants, such as talc or magnesium stearate, and optionally stabilizers. In soft capsules, the active ingredient is preferably dissolved or suspended in suitable liquid excipients, such as fatty oils, paraffin oil or liquid polyethylene glycols or fatty acid esters of ethylene or propylene glycol, to which stabilizers and detergents, for example of the polyoxyethylene sorbitan fatty acid ester type, may also be added.

In the case of combinations with one or more other active ingredients, a fixed combination of two or more components or two or more independent formulations (e.g. in a kit of part) are prepared as described above, or the other active ingredients are used in standard formulations that are marketed and known to the person of skill in the art, and the compound of the present invention and any other chemotherapeutic are administered at an interval that allows for a joint, especially a parallel, additional or preferably synergistic effect in the treatment of a proliferative disease, especially a leukaemia (especially as defined above).

The doses of chemotherapeutics to be combined with a long-acting beta-2 adrenoreceptor agonist are, for example, those used in standard treatment known in the art, e.g. as described in R. T. Skeel, Handbook of Cancer Chemotherapy, Fifth Edition, Lippincott Williams & Wilkins, Philadelphia et al., 1999, or, in view of the synergism, somewhat lower doses, e.g. between 5 and 60% of the dose without combination, respectively; the dose in each case depending on the status, age, sex, weight and other relevant properties of the patient. They can be formulated separately, especially being used in known pharmaceutical compositions, preferably combined as a kit comprising pharmaceutical preparations of each active compound (kit of parts), or in fixed combination.

Some examples for preferred dosages are represented in the following:

C-abl kinase inhibitors, especially N-{5-[4-(4-methyl-piperazino-methyl)-benzoylamido]-2-methylphenyl}-4-(3-pyridyl)-2-pyrimidine-amine monomesylate, are preferably administered to a human in a dosage in the range of about 2.5 to 1500 mg/day, more preferably 5 to 900 mg/day and most preferably 400 mg/day. Unless stated otherwise herein, the compound is preferably administered in from one to four doses per day. Administration may take place for long periods of time, e.g. several years, preferably up to three months, and preferably takes place in parallel to the administration of component (b).

For pyrimidine or especially purine nucleosides, especially, daily doses may be between 60 mg/d/m² to 400 mg/m² h, e.g. during 2 to 7 days, in the case of pentostatin every two weeks. For example, in the case of fludarabine, a bolus of 5 to 11 mg/m² over 5 to 30 min, followed by a continuous infusion of 15 to 45, e.g. 30.5, mg/m² day for 48 h, preferably followed by an ara-C loading dose of 300 to 400, e.g. 390, mg/m² over 5 to 30 min followed by a continuous infusion of 40 to 110, e.g. 101 mg/m² h for 72 hours, are preferred dosages, especially in pediatric leukaemia patients. For adults leukaemia patients, the (tolerable) total dose of ara-C should not exceed 500 to 1000 mg/m²/d×3-4 days. It is highly recommended nit to use a bolus or only to use a much reduced bolus of ara-C in these patients.

In the case of one preferred embodiment of the invention, the administration of STI571 in a dosage range of 100 to 800 mg/day (daily for a longer period of time, e.g. 3 months) as component (a) takes place orally in parallel to an administration of Fludararabine which in turn is administered in parallel or preferably before (e.g. 1 to 2 days before) administration of ara-C (together component (b), e.g. for two days before ara-C is administered, e.g. using the regimen of Fludarabine and Ara-C administration described as preferred in the preceding paragraph, especially in the case of acute leukaemia (especially T-lymphoblastic leukaemia).

In the case of another preferred embodiment of the invention, the administration of STI571 in a dosage range of 100 to 800 mg/day (daily for a longer period of time, e.g. 3 months) as component (a) takes place orally In parallel to an administration of idarubicin in a preferred dosage as provided in the following paragraph, with parallel administration of ara-C in a prerferred dosage provided in the preceding paragraph.

Among the topoisomerase II inhibitors, doxorubicin may be administered to a human in a dosage range varying from about 10 to 100 mg/m² day, e.g. 25 or 75 mg/m² day, e.g. as single dose; epirubicin may be administered to a human in a dosage range varying from about 10 to 200 mg/m² day; idarubicin may be administered to a human in a dosage range varying from about 0.5 to 50 mg/m² day, e.g. 8 mg/m² day during three days; and mitoxantrone may be administered to a human in a dosage range varying from about 2.5 to 25 mg/m² day, e.g. 10-14 mg/m² day during 5 to 8 days.

Fadrozole may be administered orally to a human in a dosage range varying from about 0.5 to about 10 mg/day, preferably from about 1 to about 2.5 mg/day. Exemestane may be administered orally to a human in a dosage range varying from about 5 to about 200 mg/day, preferably from about 10 to about 25 mg/day, or parenterally from about 50 to 500 mg/day, preferably from about 100 to about 250 mg/day. If the drug shall be administered in a separate pharmaceutical composition, it can be administered in the form disclosed in GB 2,177,700. Formestane may be administered parenterally to a human in a dosage range varying from about 100 to 500 mg/day, preferably from about 250 to about 300 mg/day. Anastrozole may be administered orally to a human in a dosage range varying from about 0.25 to 20 mg/day, preferably from about 0.5 to about 2.5 mg/day. Aminogluthemide may be administered to a human in a dosage range varying from about 200 to 500 mg/day. Tamoxifen citrate may be administered to a human in a dosage range varying from about 10 to 40 mg/day. Vinblastine (not highly recommended as secondary malignancies may occur) may be administered to a human in a dosage range varying from about 1.5 to 10 mg/m² day. Vincristine sulfate may be administered parenterally to a human in a dosage range varying from about 0.025 to 0.05 mg/kg body weight*week. Vinorelbine may be administered to a human in a dosage range varying from about 10 to 50 mg/m² day. Etoposide phosphate may be administered to a human In a dosage range varying from about 25 to 115 mg/m² day, e.g. 56.8 or 113.6 mg/m² day. Teniposide may be administered to a human in a dosage range varying from about 75 to 150 mg about every two weeks. Paclitaxel may be administered to a human in a dosage range varying from about 50 to 300 mg/m² day. Docetaxel may be administered to a human in a dosage range varying from about 25 to 100 mg/m² day. Cyclophosphamide may be administered to a human in a dosage range varying from about 50 to 1500 mg/m² day. Melphalan may be administered to a human in a dosage range varying from about 0.5 to 10 mg/m² day. 5-Fluorouracil may be administered to a human in a dosage range varying from about 50 to 1000 mg/m² day, e.g. 500 mg/m² day. Capecitabine may be administered to a human in a dosage range varying from about 10 to 1000 mg/m² day. Gemcitabine hydrochloride (not highly recommended as secondary malignancies may occur) may be administered to a human in a dosage range varying from about 1000 mg/week. Methotrexate may be administered to a human in a dosage range varying from about 5 to 500 mg/m² day. Topotecan may be administered to a human in a dosage range varying from about 1 to 5 mg/m² day. Irinotecan may be administered to a human in a dosage range varying from about 50 to 350 mg/m² day. Carboplatin may be administered to a human in a dosage range varying from about 200 to 400 mg/m² about every four weeks. Cisplatin may be administered to a human in a dosage range varying from about 25 to 75 mg/m² about every three weeks. Oxaliplatin may be administered to a human in a dosage range varying from about 50 to 85 mg/m² every two weeks. Alendronic acid may be administered to a human in a dosage range varying from about 5 to 10 mg/day. Clodronic acid may be administered to a human e.g. in a dosage range varying from about 750 to 1500 mg/day. Etridonic acid may be administered to a human in a dosage range varying from about 200 to 400 mg/day. Ibandronic acid may be administered to a human in a dosage range varying from about 1 to 4 mg every three to four weeks. Risedronic acid may be administered to a human in a dosage range varying from about 20 to 30 mg/day. Pamidronic acid may be administered to a human in a dosage range varying from about 15 to 90 mg every three to four weeks. Tiludronic acid may be administered to a human in a dosage range varying from about 200 to 400 mg/day. Trastuzumab may be administered to a human in a dosage range varying from about 1 to 4 mg/m² week. Bicalutamide may be administered to a human in a dosage range varying from about 25 to 50 mg/m² day.

Tyrphostins, especially Adaphostin, are preferably administered to a warm-blooded animal, especially a human in a dosage in the range of about 1 to 6000 mg/day, more preferably 25 to 5000 mg/day, most preferably 50 to 4000 mg/day. Unless stated otherwise herein, the compound is preferably administered from one to 5, especially from 1 to 4 times per day.

Components (a) and (b) can be prepared according to methods that are known in the art, e.g. as described in any of the references quoted herein, and/or they are commercially available. The most preferred combination partner (a), STI571, can be prepared and administered as described in WO 99/03854.

### EXAMPLES

The following Examples serve to illustrate the Invention without limiting the scope thereof:

Materials and Methods: The CCRF-CEM/0 human leukaemia cell line is obtained from DCT, Tumor Bank, NCI, NIH, Fredrick, Md. The CEM/ara-C/I/ASNase cell line (drug resistant to both ara-C and L-asparaginase) is developed by consecutive treatment with several high doses of ara-C and is partially resistant to ara-C. In short, the ara-C resistant lines are developed in our laboratory by exposure of the CEM/0 (wild-type) line to either 0.1 or 1 uM ara-C for 24 hours. After the incubation, the surviving cells are washed and plated in soft-agar 200 to 400 cells per 10 cm plate for cell colony growth. The process of treatment is repeated in selective colonies once or twice with one-log higher ara-C concentration (1 to 10 uM) for 24 hours followed by plating in soft-agar. In addition, CEM cell clones isolated from soft-agar, are cultured in enriched RPMI-1640 (10% FCS+1% amino acids+1% HEPES buffer) and daily cell counts are performed in an aliquot cell culture using a Coulter counter and a microscope/hemocytometer. After checking for viability with the Trypan-exclusion test, cell counts are plotted over time. Once growth has begun the kinetics (the slope with which the cell lines grow) are superimposable to the parent cell line, suggesting that the duplication half-life, and by extrapolation the duration of the cell cycles, has not changed. Time delays in starting the log-linear growth of these cell cultures Is related to the degree of ara-C-resistance. The cells treated 3 times with low concentrations of ara-C have virtually no time delay in growth and are many orders of magnitude resistant to the drug from 2-fold to >1E8-fold resistance (CEM/ara-C/I monoclonal clone) (Martin-Aragon S., et al., Anticancer Res., 20:139-150, 2000). No further ara-C treatments are imposed upon these cell lines and they appear to be maintaining their relative degree of ara-C resistance independent of the duration or the year of treatment (permanent drug resistant clones due to epigenetic effects on DNA hypermethylation by ara-C). The sensitivity to ara-C in the cell line mentioned above is about less than 1% of the wild type CEM/0 cell line (Yee et al., Am. Assoc. Cancer Res. 34, 416 (Abstract # 2484) and Antonsson et al., Cancer Research 47:3672-8 (1978)). Finally, there is selection of the double resistant clone CEM/ara-C/I/ASNase that is further resistant against asparaginase (Capizzi II regimen) by treating the CEM/ara-C/I clone with 0.5 to 1 IU/ml (therapeutic level in leukemic patients) native E coli saparaginase for 24 h. The cells are washed and plated in soft-agar for colonies to develop. From the resulting clones, one Is the CEM/ara-C/I/ASNase clone used in the subsequent experiments (see also Majlessipour et al., Anticancer Res. 21, 11-22 (2001). Pharmacological and enzymatic deoxycytidine kinase (dCk) determinations are performed in the monoclonal derived culture. These are conducted to determine sensitivity to ara-C and the relative percentage of dCk activity as compared with wild-type CEM/0 cells as described in Antonsson et al., Cancer Res. 47: 3672-8 (1987) and Avramis et al., Cancer Res. 49: 241-7 (1989).

The IC₅₀ of test compounds (STI571, ara-C, Fludarabine, Idarubicin) are determined against CEM/0 or CEM/ara-C/I/ASNase. 24-well plates (2 ml/well), Costar, Mark II, No. 3424) are used for determining the IC₅₀ values. The compounds are dissolved in dimethyl sulfoxide (DMSO) and the final concentration does not exceed 1%. The drug solutions are sterilized through 0.22 μm×13 mm Millipore filters (Millipore GSWP). The stock solutions (10⁻² M) are diluted using the enriched RPMI 1640 growth medium. Appropriate controls are run to account for any effect of DMAO. Each well receives 0.9 ml of the cell suspension containing 2×10⁵ cells. The treatment wells receive 0.1 ml of culture medium, RPMI with less than 1% DMSO containing the drug STI 571 in amounts so that when q.s. is added to 1 ml (0.9+0.1 ml) it will achieve the desired concentration of 10 or 0.1 μM in 1 ml cell suspension. Each remaining drug concentration (10⁻⁴ to 10⁻⁹ M) is plated in triplicate and incubated at 37° C. in an atmosphere with 95% air and 5% CO₂ for 48 h for Fludarabine+STI571 followed by ara-C or for 72 h for Idarubicine+ara-C, STI571+Fludarabine+ara-C or Idaruicine (at lower drug concentrations) or any other combination. The cells then are counted on a Coulter Counter after the desired incubation period. In addition, MTT assay is performed. The results are evaluated in an aliquot of the cells as percent of control after correcting for cell viability by the trypan blue dye-exclusion test.

Briefly, the constant ratio of “Drug A” (any single drug or combination of two drugs with established synergism) and “Drug B” (any single drug or combination of two drugs with established synergism) are used for examination. In one method, all possible drug ratios between Drug A and Drug B (if one or both are drug combinations, they are represented as one drug in final calculation) are examined. The Median Effect Principle (MEP) examines only Drug A or Drug B and their diagonal combination under a constant ratio, e.g. control (C), 0.01, 0.05, 0.1, 0.5, 1 μM or nM units and 0.01:0.01, 0.05:0.05, 0.1:0.1, 0.5:0.5 and 1:1 for combination ratio of 1:1. The ratio Is constant, but not only 1:1, it may also be 1:10 or 10:1 and the like.

Two plates represented by the following table (each cell corresponding to a well) are used, as an example:

| Control | A 0.01 | B 0.01 | B 0.05 | B 0.1 | B 0.5 | B 1.0 | Control |
|---|---|---|---|---|---|---|---|
| well | | | | | | | well |
| Control | A 0.02 | A 0.01 + B | A 0.01 + B | A 0.01 + B | A 0.01 + B | A 0.01 + B | Control |
| well | | 0.01 | 0.05 | 0.1 | 0.5 | 1.0 | well |
| Control | A 0.05 | A 0.05 + B | A 0.05 + B | A 0.05 + B | A 0.05 + B | A 0.05 + B | Control |
| well | | 0.01 | 0.05 | 0.1 | 0.5 | 1.0 | well |
| Control | A 0.2 | A 0.1 + B | A 0.1 + B | A 0.1 + B | A 0.1 + B | A 0.1 + B | Control |
| well | | 0.01 | 0.05 | 0.1 | 0.5 | 1.0 | well |
| Controll | A 0.5 | A 0.5 + B | A 0.5 + B | A 0.5 + B | A 0.5 + B | A 0.5 + B | Control |
| well | | 0.01 | 0.05 | 0.1 | 0.5 | 1.0 | well |
| Controll | A 1.0 | A 1.0 + B | A 1.0 + B | A 1.0 + B | A 1.0 + B | A 1.0 + B | Control |
| well | | 0.01 | 0.05 | 0.1 | 0.5 | 1.0 | well |

A: Drug A, concentration (number after A) given in μM or nM
B: Drug B, concentration (number after A) given in μM or nM

This represents two 24-well plates, which are used in triplicate (independent) determinations. Additional plates are set up with higher or mostly lower drug concentrations when the drug combination is found to be very effective in cell killing. In addition, the time of drug incubation can be altered (e.g. lower times if high cytotoxicity is found). Advantageously, the cell kill lies over 50%, but not all values can be over 50% due to the limitations of the program estimating ED₅₀ values from values only below (0 to 49) or above 50% (51 to 99%) cell killing of control. Drug combinations acting very fast in Inducing apoptosis (e.g. STI+Fludara+ara-C) are stopped at 48 h, others are continued until 72 h (see figures). No cell cultures are maintained for more than 72 h (as then the growth media nutrients become limiting). The data are analysed via the MEP standard program.

A variation of this model is represented by the following table in which each 24-well plate is used for 6 concentrations of each drug or drug combination in triplicate plus control (6×). The third plate is used for the constant ratio of the drug combination Drug A plus Drug B, two each under Drug A and Drug B name. The results are handled in the same manner by MEP.

| | | | | | | | | | | | |
|---|---|---|---|---|---|---|---|---|---|---|---|
| control | A | A | A | Control | B | B | B | Control | A | A | A |
| | 0.01 | 0.01 | 0.01 | | 0.01 | 0.01 | 0.01 | | 0.01 + B | 0.01 + B | 0.01 + B |
| | | | | | | | | | 0.01 | 0.01 | 0.01 |
| Control | A | A | A | Control | B | B | B | Control | A | A | A |
| | 0.05 | 0.05 | 0.05 | | 0.05 | 0.05 | 0.05 | | 0.05 + B | 0.05 + B | 0.05 + B |
| | | | | | | | | | 0.05 | 0.05 | 0.05 |
| Control | A | A | A | Control | B | B | B | Control | A | A | A |
| | 0.1 | 0.1 | 0.1 | | 0.1 | 0.1 | 0.1 | | 0.1 + B | 0.1 + B | 0.1 + B |
| | | | | | | | | | 0.1 | 0.1 | 0.1 |
| Control | A | A | A | Control | B | B | B | Control | A | A | A |
| | 0.5 | 0.5 | 0.5 | | 0.5 | 0.5 | 0.5 | | 0.5 + B | 0.5 + B | 0.5 + B |
| | | | | | | | | | 0.5 | 0.5 | 0.5 |
| Control | A | A | A | Control | B | B | B | Control | A | A | A |
| | 1.0 | 1.0 | 1.0 | | 1.0 | 1.0 | 1.0 | | 1.0 + B | 1.0 + B | 1.0 + B |
| | | | | | | | | | 1.0 | 1.0 | 1.0 |
| Control | A 5 | A 5 | A 5 | Control | B 5 | B 5 | B 5 | Control | A 5 + B 5 | A 5 + B 5 | A 5 + B 5 |

A: Drug A, concentration (number after A) given in μM or nM
B: Drug B, concentration (number after A) given in μM or nM

The drugs/drug combinations used are given in detail in the Examples below.

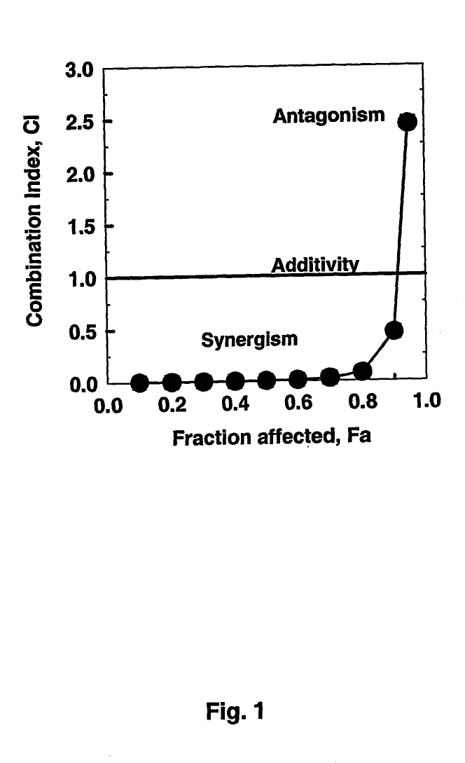

Probit analysis is performed to obtain the ED₅₀ values. The number of viable cells per well over a 2 to 3 day period versus time is plotted. The isobologram and median-effect principle are used to obtain the ED₆₀ values (see Avramis et al., Cancer Res. 49: 241-7 (1989); and Chou, The median effect principle and the combination index for quantitation of synergism and antagonism, in: Chou, T. C., and Rideout, D. C. (eds.), “Synergism and antagonism in chemotherapy”, cademic Press, Orlando (1991), pp 61-90). Median effect plots (see FIG. 1 to FIG. 4) are prepared by plotting CI on Y-axis and fₐ (fraction affected) on X-axis for mutually nonexclusive and/or exclusive case of synergism.

Isobologram method: The isobologram method involves the use of the following equation:
CI=(A_{c}/Aₑ)+(B_{c}/Bₑ)
where CI is the combination index, Aₑ and Bₑ are the dose of Drug A and Drug B alone that are are required to inhibit a system by x % (e.g. 50%) and A_{c} and B_{c} are the concentrations of compounds in combination that inhibit x % of the system.

Median effect equation: The median-effect principle involved the use of the following equation:
fₐ/fᵤ=(D/Dₘ)^{m}
where D is the dose, fₐ is the fraction of the systems affected by dose D, fᵤ is the fraction of the system unaffected by dose D, Dₘ is the dose required to produce the median effect (analogous to the IC₅₀), m is the Hill-type coefficient signifying sigmoidicity of the dose-effect curve, fₐ+fᵤ=1, D=Dₘ[fₐ/(1−fₐ)]^{1/m}, and log(fₐ/fᵤ)=mlog(D)+mlog(Dₘ).

For calculation of the CI for mutually exclusive drugs (having the same mechanism of action):
CI=(D)₁/(Dₓ)₁+(D)₂/Dₓ)₂
For calculation of the CI (combination Index) for mutually non-exclusive drugs (having different mechanisms of action):
CI=(D)₁/Dₓ)₁+(D)₂/Dₓ)₂+(D)₁×(D₂/(Dₓ)₁×(Dₓ)₂
For mutually exclusive or nonexclusive drugs, when CI<1, synergism is indicated; when CI=1, additivity is indicated; and when CI>1, antagonism is indicated.

In the tables provided below, any value of under “synergism” is regarded as additive if between 1 and 2, above 2.5 as moderately synergistic, and above 4 to 5 as highly synergistic. Any value below 1 is considered as antagonistic (meaning that the combined drugs are attacking the same target or that (as often found when using high drug concentrations) there is a saturable step in inducing cytotoxicity.

### Example 1

### N-{5-[4-(4-methyl-piperazino-methyl)-benzoylamido]-2-methylphenyl}-4-(3-pyridyl)-2-pyrimidine-amine Monomesylate Salt (STI571) in Combination with Fludarabine and Cytosine Arabinoside (ara-C)—Effect on CEM/0 Cells

If STI571 and after 4 h Fludarabine and ara-C are administered to CEM/O-cells for a total treatment duration of 48 h, the Combination Index (CI)-Fraction affected relation represented graphically in FIG. 1 is obtained. Assuming mutually non-exclusive effects of the drugs, the following synergistic factors are obtained for the triple combinations over the combination pairs Fludarabine plus ara-C:

| Effective dose | Synergism |
|---|---|
| ED₅₀ | 14.8-fold |
| ED₇₀ | 22.8-fold |
| ED₉₀ | 1.1-fold |

From these data it follows that synergism is found between STI571 and Fludarabine and ara-C at ED₅₀ and ED₇₀, but not at ED₉₀.

### Example 2

### N-{5-[4-(4-methyl-piperazino-methyl)-benzoylamido]-2-methylphenyl}-4-(3-pyridyl)-2-pyrimidine-amine Monomesylate Salt (STI571) in Combination with Fludarabine and Cytosine Arabinoside (ara-C) with Fludarabine Given First—Effect on CEM/0 Cells

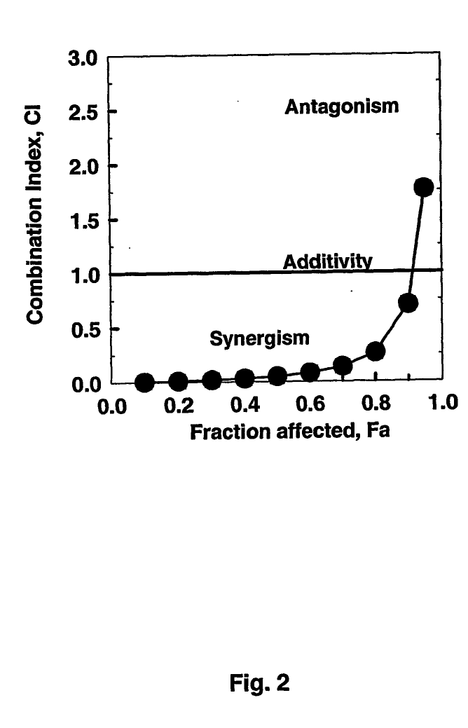

If Fludarabine and after 4 h STI571 and ara-C are administered to CEM/0-cells for a total treatment duration of 48 h, the Combination Index (CI)—Fraction affected relation represented graphically in FIG. 2 is obtained. Assuming mutually non-exclusive effects of the drugs, the following synergistic factors are obtained for the triple combinations over the combination pairs Fludarabine plus ara-C:

| Effective dose | Synergism |
|---|---|
| ED₅₀ | 10.6-fold |
| ED₇₀ | 3.8-fold |
| ED₉₀ | 0.7-fold |

It follows that, when compared with Example 1, less drug synergism is found when Fludarabine treatment preceeds STI treatment by 4 hours at ED₅₀ and ED₇₀—at ED₉₀ no synergism is found.

### Example 3

### N-{5-[4-(4-methyl-piperazino-methyl)-benzoylamido]-2-methylphenyl}-4-(3-pyridyl)-2-pyrimidine-amine Monomesylate Salt (STI571) in Combination with Fludarabine and Cytosine Arabinoside (ara-C) with Fludarabine Given First—Effect on Resistant CEM/ara-C/I/ASNase-0.5-2 Cells

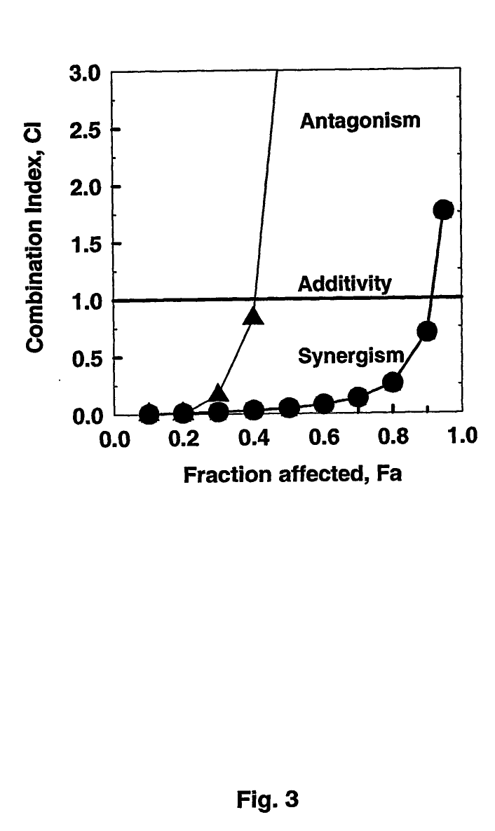

In order to compare the effects on wild type CEM/0 cells with those on ara-C resistant CEM/ara-C/I/ASNase-0.5-2 cells, the effects of a combination of first Fludarabine, then after 4 h STI571 and ara-C addition are determined. FIG. 3 (triangles) shows the CI/Fa plot for this experiment (for comparison, the data from FIG. 2 are also included as circles). Calculating the synergism, a 111.2-fold effect is found for the ED₅₀, showing drug synergism in the drug resistant cell line.

### Example 4

### N-{5-[4-(4-methyl-piperazino-methyl)-benzoylamido]-2-methylphenyl}-4-(3-pyridyl)-2-pyrimidine-amine Monomesylate Salt (STI571) in Combination with Idarubicin and ara-C—Effect on CEM/0 Cells

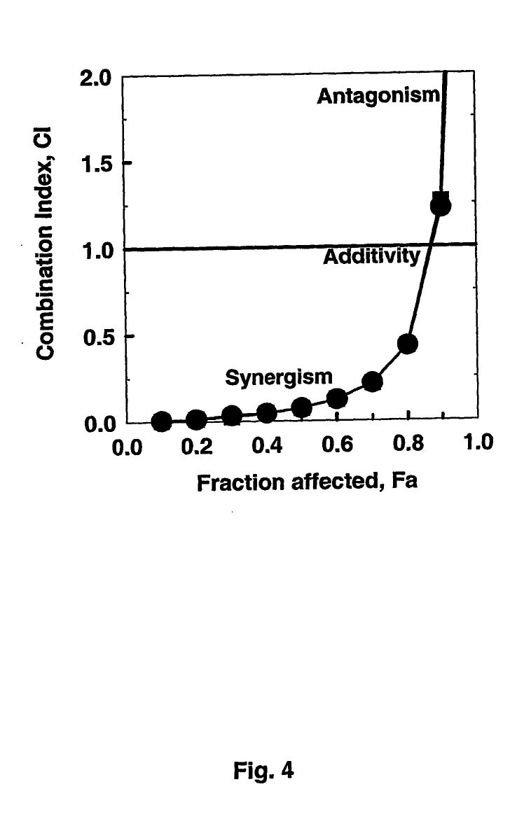

If STI571 and Idarubicin plus ara-C are administered to CEM/0-cells for a total treatment duration of 72 h, the Combination Index (Cl)—Fraction affected relation represented graphically in FIG. 4 is obtained. Assuming mutually non-exclusive effects of the drugs, the following synergistic factors are obtained for the triple combinations over the combination pairs Fludarabine plus ara-C:

| Effective dose | Synergism |
|---|---|
| ED₅₀ | 4.9-fold |
| ED₇₀ | 6.3-fold |
| ED₉₀ | 10.0-fold |

Thus, the already highly synergistic regimen of Idarubicin plus ara-C potentiates the effect of STI571 with a remarkable Increase in drug synergism especially at ED₉₀.

What follows from the examples above Is that STI571 can be of use not only in Ph⁺—but also in other leukaemias in combination with other cytotoxic combination regimens.

### Example 5

### Outlining of Clinical Trials with STI/Fludarabine/ara-C or STI/Idarubicin/ara-C Combinations

Clinical trials are conducted with Acute Myeloid Leukaemia (AML), Acute Lymphoblastic Leukaemia (ALL) or especially Chronic Myeloid Leukaemia (CML) patients. The required permissions are obtained.

Variant a) For the treatment with the STI571/Fludarabine/ara-C combination, STI571 is administered daily in an escalating way with doses of 200, 400 or 600 mg/day orally, starting on day 1.

Dose Level 1:

Day 1: 200 mg STI are administered orally, and after 30 min a loading bolus of Fludarabine (10 mg/m²) is administered during 15 min followed by continuous infusion (CI) of 30 mg/m²/24 h for 48 h.

On day 2, again 200 mg of STI571 are administered orally and Fludarabine CI is continued with the same dose as on day 1.

On day 3 (48.1 h), again 200 mg of STI571 are administered orally, and a loading bolus of ara-C (75% of the maximum tolerated dose (=MTD, e.g. 390 mg/m²/day in children)) is administered, followed by its continuous infusion at 75% of the MTD (MTD=100 mg/m²/day in children) for 24 h.

On day 4, again 200 mg of STI571 are administered orally and ara-C CI is continued at the dose given on day 3.

On day 5, again 200 mg of STI571 is administered orally and ara-C CI is continued at the dose given on day 3.

On the following days, oral STI571 administration (200 mgday) is continued daily for a longer period of time (at least 30 days).

Dose level 2:

The same therapy as given under Dose level 1 is conducted, except that 400 mg STI is orally administered daily.

Dose level 3:

The same therapy as given under Dose level 1 is conducted, except that 600 mg STI is orally administered daily.

Dose level 4:

The same therapy as given under Dose level 1 is conducted, except that the dose of ara-C is now 100% of the MTD (both for the bolus and the CI).

Dose level 5:

The same therapy as given under Dose level 1 is conducted, except that the dose of STI571 is 400 mg (administered orally) and the dose of ara-C is now 100% of the MTD.

Dose level 6:

The same therapy as given under Dose level 1 is conducted, except that the dose of STI571 is 600 mg (administered orally) and the dose of ara-C is now 100% of the MTD.

For each dose level, at least 4 patients are examined in the beginning in order to make sure that at least 3 patients are evaluable at the end of each study.

The bone marrow aspirate is examined (see below) pre-therapeutically and at hour 72 (or 24 h post ara-C administration), and at day 24-28 for marrow response and MRD determination.

If evaluation does not provide any contraindications, the treatment at an appropriate level is repeated after about 28 days.

Plasma samples for ara-C steady state concentration determination are collected a 4 h after beginning of Loading bolus and continuous infusion and 20 and 48 hours after ara-C termination.

For adult patients, the dose of ara-C is reduced to 500 mg/m²/day as i.v. infusion and escalated to 750 and 1000 mg/m²/day based upon safety evaluations. Oral STI571 administration in the dosage mentioned above is continued.

Variant b) For the treatment with a STI571/Idarubicin/Fludarabine/ara-C combination, STI571 is administered daily in a dose of 200, 400 or 600 mg/day, followed on days “0”, “1” and “2” by a intravenous bolus administration of 8 mg/m²/day of Idarubicin and fludarabine administered as in variant a). In parallel, on day “0” an ara-C loading dose of 250 mg/m² is administered during 15 min, followed by 65 mg/m²/h of its continuous infusion during 72 h. Oral STI administration in the dosage mentioned above is continued. Dose escalation can be made in principle analogous to variant b).

General:

Criteria for response: A complete response (CR) is considered to have occurred in any of the following circumstances in ALL or AML patients: (a) the patient has an M1 marrow (<5% blasts) with recovery of peripheral counts (ANC≧1,000/mm³ and platelet count≧100,000/mm³); or (b) the patient has an M1 marrow without the recovery of peripheral counts prior to treatment. A partial response (PR) is considered to have occurred in any of the following circumstances: (a) the patient has an M2 marrow with recovery of peripheral counts (ANC≧1,000/mm³ and platelet count≧100,000/mm³); or (b) the patient has an M1 marrow without the recovery of peripheral counts prior to treatment. Clinical toxicity is graded according to the Common Toxicity Scale of the Division of Cancer Treatment of the National Cancer Institute. This is a I-IV scale, with IV defined as life-threatening. Specific limits for each toxicity depend on the organ system.

For bone marrow examination, two bone marrow aspirates are obtained, one before STI571 treatment start (control) and the second on day 3 post treatment. A third aspirate should be drawn on day 24 to 28 for evaluation of CR, PR or No Response (NR). Bone marrow aspirates are obtained under local anesthesia from treated patients, placed in heparinized tubes and placed In an ice bath. This limitation is applied because the blast cells are separated, extracted and tested within 1-2 h after the specimens are obtained from the patients, independent of the time the specimens are drawn, in order to maintain intact enzymatic activities of the leukaemia cells. The pre-treatment specimens are tested ex vivo with two concentrations of ara-C, 200 μM and 1 mM, and the cells are extracted with perchloric acid for ara-CTP determination.

Determination of ara-C, Fludarabine, Idarubicin and STI571 blood levels follows standard methods (see, e.g., Avramis et al., Clin. Cancer Res. 4, 45-52, 1998; and Dinndorf et al., J. Clin. Oncol. 15(8), 2780-85, 1997).

1. A combination of (a) an ATP-competitive inhibitor of c-abl kinase activity and (b) with (b) two or more other antineoplastic agents for simultaneous, separate or sequential use.

2. The combination according to claim 1 where the ATP-competitive inhibitor of c-abl kinase activity (a) is N-{5-[4-(4-methyl-piperazino-methyl)-benzoylamido]-2-methylphenyl}-4-(3-pyridyl)-2-pyrimidine-amine, or a pharmaceutically acceptable salt thereof.

3. The combination according to claim 1 where the ATP-competitive inhibitor of c-abl kinase activity (a) is N-{5-[4-(4-methyl-piperazino-methyl)-benzoylamido]-2-methylphenyl}-4-(3-pyridyl)-2-pyrimidine-amine, or a pharmaceutically acceptable salt thereof, and (b) the two or more antineoplastic agents are selected from purine nucleoside analogs and topoisomerase II inhibitors which are independently present in free form or as pharmaceutically acceptable salts.

4. The combination according to claim 1 where the ATP-competitive inhibitor of c-abl kinase activity (a) is N-{5-[4-(4-methyl-piperazino-methyl)-benzoylamido]-2-methylphenyl}-4-(3-pyridyl)-2-pyrimidine-amine, or a pharmaceutically acceptable salt thereof, and (b) the two or more antineoplastic agents are selected from Idarubicine, Fludarabine and ara-C which are independently of each other present in free form or as pharmaceutically acceptable salts.

5. A method of treating a warm-blooded animal suffering from a proliferative disease, comprising administering to said animal a combination which comprises (a) an ATP-competitive inhibitor of c-abl kinase activity and (b) two or more other antineoplastic agents, where the active compounds falling under (a) and/or (b) are independently of each other in free form or in the form of pharmaceutically acceptable salts, in a dose that is pharmaceutically effective in the treatment of said disease.

6. The method according to claim 5 where component (a) is (N-{5-[4-(4-methyl-piperazino-methyl)-benzoylamido]-2-methylphenyl}-4-(3-pyridyl)-2-pyrimidine-amine, or a pharmaceutically acceptable salt thereof.

7. The method according to claim 5 where component (a) is N-{5-[4-(4-methyl-piperazino-methyl)-benzoylamido]-2-methylphenyl}-4-(3-pyridyl)-2-pyrimidine-amine, or a pharmaceutically acceptable salt thereof, and component (b) is a combination of two or more of the compounds selected from purine nucleoside analogs and topoisomerase II inhibitors, independently in free form or as pharmaceutically acceptable salts.

8. The method according to claim 5 where component (a) is N-{5-[4-(4-methyl-piperazino-methyl-benzoylamido]-2-methylphenyl}-4-(3-pyridyl)-2-pyrimidine-amine, or a pharmaceutically acceptable salt thereof, and component (b) includes two or more of the compounds selected from Idarubicine, Fludarabine and ara-C which are independently of each other present in free form or as pharmaceutically acceptable salts.

9. The method according to claim 5 where the proliferative disease is a leukaemia.

10. A pharmaceutical composition comprising a combination of (a) an ATP-competitive inhibitor of c-abl kinase activity with (b) two or more other antineoplastic agents and optionally at least one pharmaceutically acceptable carrier for the delay of progression or treatment of a proliferative disease in a warm-blooded animal, especially a human, in need of such treatment.

11. The pharmaceutical composition according to claim 10 where component (a) is (N-{5-[4-(4-methyl-piperazino-methyl)-benzoylamido]-2-methylphenyl}-4-(3-pyridyl)-2-pyrimidine-amine, or a pharmaceutically acceptable salt thereof.

12. The pharmaceutical composition according to claim 10 where component (a) is (N-{5-[4-(4-methyl-piperazino-methyl)-benzoylamido]-2-methylphenyl}-4-(3-pyridyl)-2-pyrimidine-amine, or a pharmaceutically acceptable salt thereof, and component (b) includes two or three, preferably two, further antineoplastic agents selected from purine nucleoside analogs and topoisomerase II inhibitors, or pharmaceutically acceptable salts thereof.

13. The pharmaceutical composition according to claim 10 where component (a) is (N-{5-[4-(4-methyl-piperazino-methyl)-benzoylamido]-2-methylphenyl}-4-(3-pyridyl)-2-pyrimidine-amine, or a pharmaceutically acceptable salt thereof, and component (b) includes two or more of the compounds selected from Idarubicine, Fludarabine and ara-C which are independently of each other present In free form or as pharmaceutically acceptable salts.

14. A commercial package comprising (a) an ATP-competitive inhibitor of c-abl kinase activity and (b) two or more other antineoplastic agents, where the active compounds falling under (a) and/or (b) are independently of each other in free form or in the form of pharmaceutically acceptable salts, for simultaneous, chronically staggered or (less prefereably) separate use in the delay of progression or treatment of a proliferative disease.
